(19) United States
(12) Patent Application Publication
(10) Pub. No.: US 2008/0183102 A1
(43) Pub. Date: Jul. 31, 2008

(54) Urine collection device

(76) Inventor: Gary Dunkin, Glen Ridge, NJ (US)

Correspondence Address: RALPH T. LILORE, 60 ROUTE 46 EAST, FAIRFIELD, NJ, 07004, US

(21) Appl. No.: 12/008,754

(22) Filed: Jan. 14, 2008

Related U.S. Application Data
(60) Provisional application No. 60/897,347, filed on Jan. 25, 2007.

(51) Int. Cl. B65D 33/00; A61B 5/00

(52) U.S. Cl. 600/580; 600/573

(57) ABSTRACT

A urine collection device made of a flexible, liquid-impermeable vessel or collection bag in which there is a urine entry site and which is sealed on all other sides, against loss of urine. The vessel may be a plastic bag, hermetically sealed against entry or exit of liquid except for a liquid entry site to allow the inflow of urine from the patient into the bag. The bag may be propped up interiorly by the inclusion of a lightweight, flexible filler agent, such as a soft, interstitial material, preferably a non-absorbent or only partly absorbent material, to give depth to the device and accommodate, more directly, entry of liquid into the collection device. While the interstitial material is preferred, a solid flexible scaffolding structure, or individual spacers may be employed to keep the bag propped open.

### CROSS REFERENCE TO RELATED APPLICATIONS

This application claims the benefit of U.S. Provisional Application 60/897,347, filed Jan. 25, 2007.

### BACKGROUND OF THE INVENTION

### Field of the Invention

This invention relates to urine collection devices and more particularly to devices for urine collection from infants and other patients unable to cooperate in voluntarily supplying urine samples.

It is often necessary to collect urine for testing from patients unable to voluntarily supply a urine sample. Notably among these are infants. Currently, the manner in which urine is collected from those individuals leaves much to be desired.

Since infants are unable to cooperate in supplying a urine sample, steps have to be taken to ensure that when the infant urinates, the urine is collected without cooperation from the infant and without immediate intervention by a third party to collect the urine as it is leaves the body. In many cases, a septic collection is necessary.

A current device, used mostly for infants, comprises a barrier layer attached to the patient's skin on one side and having a discharge tube attached to a collection bag. The barrier layer is adhesively attached to the skin of the infant around the genital area to act as a blockade against loss of urine to surrounding, uncovered areas prior to collection.

The adhesive edges act to contain the urine, leaving only the discharge tubing as the conduit for unimpeded flow of urine away from the patient's body and into the collection bag where it remains until it is collected by concerned personnel.

This device and others like it are highly undesirable as currently used in the field. In order for the adhesive at the edges to form a tight seal, it must be firmly adherent to the skin to prevent dislocation by the action of a moving infant which may cause individual areas of leakage. This very tightness causes extreme pain to the infant when pulled off the skin after use. In some cases, areas of skin may even be inadvertently removed in the process.

### BRIEF SUMMARY OF THE INVENTION

The present invention eliminates these and other disadvantages of the prior art devices. In the present invention, adherence to the skin is not required, as will be seen below.

In its simplest form, the device may be seen as an insert in the diaper of the patient and held in place over the genital area. The collection device insert comprises, in general, a flexible, liquid-impermeable vessel or collection bag in which there is a urine entry site and which is sealed on all other sides, against loss of urine. The collection device may be, for example, a plastic bag, hermetically sealed against entry or exit of liquid except for a liquid entry site to allow the inflow of urine from the patient into the bag. While the device may be comprised of simply a flattened bag with the liquid entry site, urine collection is facilitated if the bag is propped up interiorly by the inclusion of a lightweight, flexible filler agent which fits into the bag lifting the upper layer of the bag away from the lower layer thus providing volume to the bag to receive urine. The preferred agent is a soft, interstitial material, preferably a non-absorbent or only partly absorbent material, to give depth to the device and accommodate more directly entry of liquid into the collection device. While the interstitial material is preferred, a solid flexible scaffolding structure, or individual spacers may be employed to keep the bag propped open.

In use, the device is applied over the urethra with the urine entry site in registration therewith. The applied diaper will serve to hold the collection device in place. For various reasons, the patient is checked periodically to see if urine has been collected in the device. When enough urine has been collected, the device is removed and the contents emptied into a suitable vessel for subsequent processing and testing.

There are a variety of aspects to the foregoing device and embodiments which are more conveniently explained by reference to the drawings herein and the detailed description accompanying them.

### BRIEF DESCRIPTION OF THE DRAWINGS

FIG. 1 is a perspective view of an embodiment of a simple form of the collection device with a single large urine receiving site and without interstitial material or any other “propping up” material.

FIG. 2 is a perspective view of the device of FIG. 1 containing an interstitial material as an envelope bulking filler which also acts as an ancillary urine reservoir.

FIG. 3 is a perspective view of an embodiment of the invention wherein the urine receiving site is comprised of a plurality of voids in the envelope of the collection device.

FIG. 4 is a perspective view of an embodiment of the device of the invention wherein an adhesive backing is provided for securing the collection device to a diaper.

FIG. 5 is a perspective view of FIGS. 1, 2, and 4 where the edges of the urine receiving site are sealed to the filler material.

FIG. 6 is a perspective view of an embodiment of the invention showing a discharge area for emptying of contents of the collection device.

FIG. 7 is a perspective view of an embodiment of the invention wherein the device is equipped with a urine discharge tube.

### DETAILED DESCRIPTION OF THE INVENTION

The invention and various embodiments thereof will be better understood by reference to the drawings.

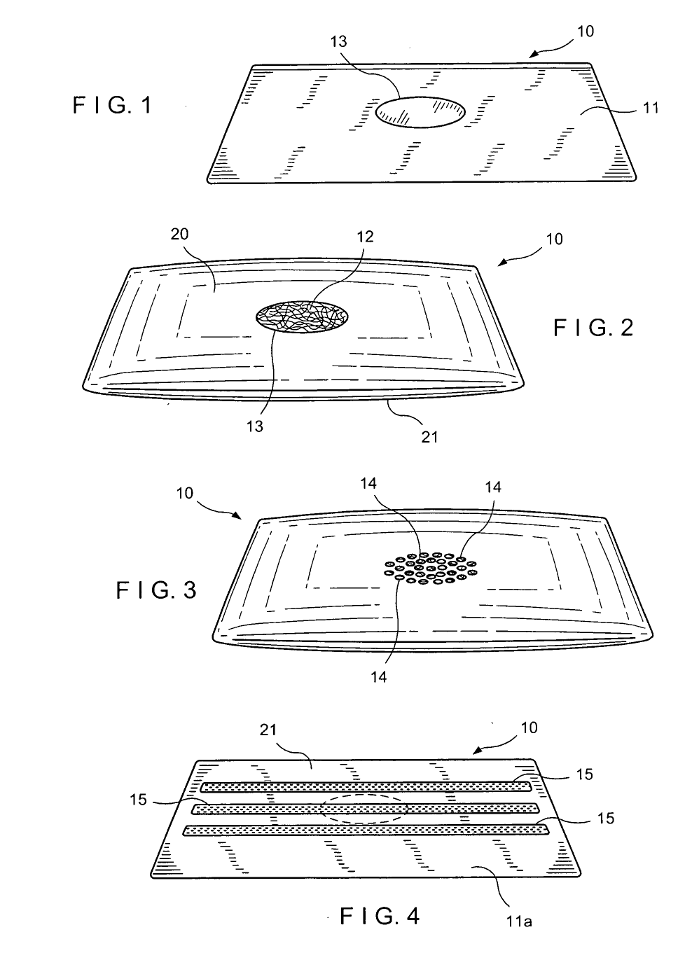

FIG. 1 shows urine collection device 10 in a simple embodiment thereof. The device comprises envelope 11 which constitutes the container or bag element of the device 10. Envelope 11 is a water-impermeable, flexible material, such as a plastic. It is preferably soft so as not to cause discomfort to the wearer. The device can be visualized as resembling a sealable plastic bag used for storage of various items such as foods, trinkets, office supplies and the like. It need not be resealable when single use is desired.

For purposes of illustration, the bag may be derived for example, from a roll of a tubular plastic, cut to the proper length size, the ends of which are sealed to provide a water tight vessel except for urine receiving site 13 which constitutes an opening in the otherwise water tight collection device 10.

The invention is quite adaptable to any size patient since the same size device can be used on a wider range of patient sizes than encountered in the adhesive device. This is in contradistinction to the adhesively affixed prior art device which is more dependent on the size of the patient.

In FIG. 2, device 10 is shown as including a fibrous, interstitial material 12 inside the bag of the device. Thus, the space between the top layer 20 and bottom layer 21 of device 10 in FIG. 2 is filled with the interstitial material 12. The material serves several purposes. First, it serves to plump up the plastic envelope 11 and thereby increases the volume available for receiving urine within the envelope over that available in an otherwise flattened envelope, thus facilitating entry of urine into the device.

Secondly, the interstices of the insert serve as a reservoir for inflowing urine. In this regard, while virtually any propping up material or interstitial material may be employed, it is preferred to use non-absorbent material or partially absorbent material to avoid the infiltration of urine into fibers that would occur with absorbent materials. Rather, the use of non-absorbent or partially absorbent fibers allows the liquid to be held in the interstices or spaces between fibers. The use of an absorbent material may require somewhat more handling in the process of emptying the collection device to process the urine.

The term “interstitial material” as used herein is meant to apply to materials which have a multiplicity of spaces in which liquids such as urine may be received and from which urine may be discharged. As examples of interstitial materials suitable in the invention there may be mentioned natural or synthetic sponges, fibrous collagen layers or sponges, fibrous natural or synthetic polymers such as polyethylene, and polypropylene in the form of batting of the type used for stuffing quilts or for packaging, layers of gauze or cotton wadding material, or the like. We prefer non-absorbent materials, by which we mean that the material used for the urine reservoir is made of fibers which themselves do not absorb the liquid. Instead, the liquid is collected within the void interstitial spaces of the fibrous structure. Batting made of synthetic fibers is also suitable for this purpose.

In FIG. 3, collection device 10 is shown as having a plurality of small openings 14 which act collectively as the urine receiving site instead of the single large receiving site 13 shown in FIGS. 1 and 2. While openings 14 are shown as small holes, they may be any size or shape desired such as slots, ellipses, ovals, or the like, and they may be in any desired pattern provided that the aggregate area of open spaces is sufficient to receive a desired amount of urine.

In FIG. 4, the device of any of those in the Figures (FIG. 1 being representative) is shown wherein the underside of the device 11a comprises strips of adhesive 15 attached to the bottom of the device. The purpose of the strips is to supply an adhesive surface to the device to provide an adhering means which enables the device to be held in place in the patient's diaper thereby avoiding movement away from the urethra.

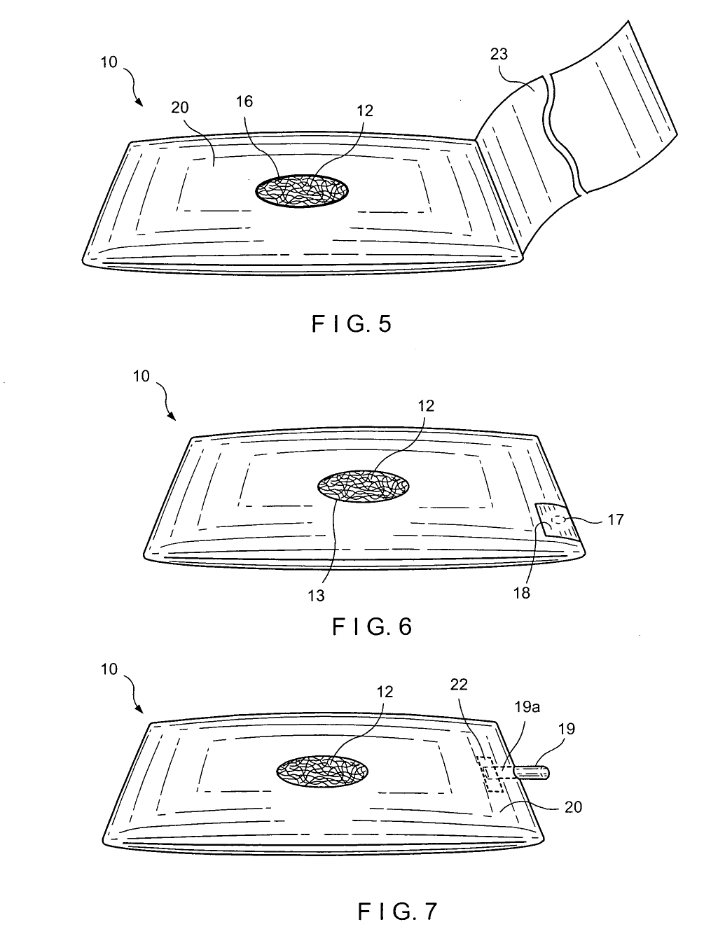

In FIG. 5, there is shown an improvement in device 10 wherein the urine receiving site 13 of FIG. 1 is shown at 16 in FIG. 5 as being sealed (heavy oval line) to the fibrous interstitial material 12 around the edges. The sealed edge serves to provide a barrier to the outflow of urine that has already been collected by inflow through urine receiving site 13. The sealing may be accomplished by spot heat welding or gluing of the edges to the bulking material. If desired, the sealed weld may be pressured against the opposing envelope layer 21 to form a quilted pattern, provided that adequate spacing remains open to the flow of urine into the device through site 13.

FIG. 5 also illustrates an additional aspect of the invention which may be applied to any other embodiments of the invention. That is, cover 20 is provided as a soft, body-compatible covering of the device 10 to provide a comfortable interface between the patient's body and the surface of device 10. This may be in the form of a simple flap as is shown in FIG. 5 at 20 or may be in the form of a sleeve or envelope encompassing the device 10. Cover 20 is a soft, porous material enabling the passage of urine into the device at receiving site 13.

FIG. 6 shows the device of the invention equipped with discharge means 17 and 18 to allow emptying of urine from the device to a testing vessel (not shown). In this case, the discharge means is nothing more than a hole 17 (shown as a dotted circle) which is taped over by tape 18. When the device is ready for emptying, the device may be held vertically with the hole 17 at the bottom, the tape 18 removed, and the contents collected in a suitable vessel. A sliced cutaway may be used instead of hole 17 or indeed if a zip-lock sealing means is employed in the device, simply sliding the zip-lock to a less than complete closure would achieve the same results. In short, there is no particular restriction on the discharge means employed.

FIG. 7 shows another discharge means in the form of a discharge tube 19. This tube may be folded under device 10 and taped in place by tape 22 during use in the manner shown on 21 and thereafter opened for discharge of urine when sufficient sample has been collected.

For ease of collection and to avoid contamination with extraneous contaminants, device 10 is preferably positioned on the patient with the discharge means in the anterior position, the opposite end of device 10 being posteriorly located. The urine receiving site is positioned over the urethra. The entire collection device is position inside the diaper which then holds the device in place.

When sufficient urine has been excreted into the device (a condition which will be determined by the patient's handlers) the device is carefully removed and held vertically to permit the liquid contents to be discharged into a suitable vessel for testing.

The device of the invention may be made by a variety of techniques well-known to those skilled in the art. There is no special procedure required and all of the steps needed to produce the product will at once be obvious to those skilled in the art of manufacturing such products. Suitably one may select an appropriately sized plastic bag, such as a polyethylene bag, with a sealable edge, provide an opening in the bag at the desired location for the urine receiving site, provide the bag with a suitable amount of the fibrous, bulking material, or other propping up material, provide the bag with sealed edges against the outflow of urine and provide the bag with discharge means. If desired, seal the edges of the urine receiving site may be sealed to the bulking interstitial material.

From the foregoing description, it can be seen that the urine collection device of the present invention comprises a collection envelope comprising a soft, flexible liquid collection reservoir which is generally adaptable and conformable to the peri-urethral anatomy of the patient, said envelope further comprising an opening for receiving voided urine from a patient, said reservoir being sealed to the outflow of collected urine therefrom except for said opening, said reservoir also comprising a propping up agent whereby the upper and lower surfaces of said envelope are separated from each other, such as an interstitial material for receiving and accommodating said collected urine, said collection device also comprising discharge means for removing the collected urine from the device.

The device may also include any one or more, in any combination, of:
a soft covering for the urine receiving site,
a plurality of openings as the urine receiving site
a soft, flexible, generally body-conformable, plastic material for the urine reservoir,
an adhesive surface preferably on the underside of the collection device, to hold the device in place on a diaper.

What is claimed is:

1. A liquid collection device which comprises a flexible, liquid-impermeable collection bag comprising an upper layer and a lower layer, a liquid entry site in one of said layers, said layers being hermetically sealed against loss of liquid on all sides except the entry site.

2. The device of claim 1 wherein said collection bag is propped up interiorly by the inclusion of a flexible filler agent in said bag whereby the upper layer of the bag is held away from the lower layer thus defining volume to the bag for receiving urine.

3. The device of claim 2 wherein said flexible filler agent comprises a soft, interstitial material.

4. The device of claim 3 wherein the liquid entry site is an opening in the collection device.

5. The device of claim 1 wherein an adhesive backing is provided on said device on the layer opposite to that of the urine entry site for securing the collection device to a diaper.

6. The device of claim 2 wherein the edges of the urine receiving site are sealed to the filler material.

7. The device of claim 1 wherein the collection bag comprises a discharge area for emptying of contents of the collection device.

8. The device of claim 7 wherein the device is equipped with a urine discharge tube.

9. The device of claim 4 comprising a plurality of small openings acting collectively as the liquid entry.

10. The device of any of claim 4 wherein the edges of the urine receiving site is sealed to the fibrous interstitial material.

11. The device of claim 1 comprising a soft, body-compatible water-permeable covering over the device to provide an interface between the patient's body and the surface of the device.

12. The device of claim 8 wherein the liquid discharge tube is adapted to be folded, taped in place on said collection bag before and during use, and thereafter openable for discharge of urine when sufficient sample has been collected.

13. A urine collection device comprising a collection envelope comprising a soft, flexible liquid collection reservoir which is generally adaptable and conformable to the peri-urethral anatomy of the patient, said envelope further comprising an opening for receiving voided urine from a patient, said reservoir being sealed to the outflow of collected urine therefrom except for said opening, said reservoir also comprising a propping up agent whereby the upper and lower layers of said envelope are separated from each other, said collection device also comprising discharge means for emptying the collected urine from the device.
